(19) United States
(12) Patent Application Publication
(10) Pub. No.: US 2016/0051415 A1
(43) Pub. Date: Feb. 25, 2016

(54) DNA COLLECTION KIT AND DNA COLLECTING METHOD USING THE SAME

(71) Applicant: TCI Gene, Inc., Taipei (TW)

(72) Inventors: Shu-Chun Kuan, Taipei (TW); Yung-Hsiang Lin, Taipei (TW); Hui-Hsin Shih, Taipei (TW); Hsueh-Yin Huang, Taipei (TW); Yueh-Ying Tsai, Taipei (TW)

(21) Appl. No.: 14/465,909

(22) Filed: Aug. 22, 2014

(51) Int. Cl. A61F 13/38; A61B 19/02; A61F 13/53; A61B 10/02; A61B 10/00

(52) U.S. Cl. CPC A61F 13/38; A61B 10/02; A61B 10/0096; A61F 13/53; A61B 19/026; A61F 2013/530802

(57) ABSTRACT

A DNA collection kit comprises a foldable collection rod, a collection bottle, and a buffer comprising ethylenediamine tetraacetic acid (EDTA) at a concentration from 20 mM to 60 mM. A method for using a DNA kit comprises the following steps: collecting DNA from a collection portion of the foldable collection rod of the DNA collection kit; breaking the foldable collection rod from the groove of the rod body to obtain a DNA-comprising collection portion; and putting the DNA-comprising collection portion into the collection bottle comprising the buffer to prolong the preservation time of the DNA, wherein the buffer comprises ethylenediamine tetraacetic acid (EDTA) at a concentration from 20 mM to 60 mM.

### BACKGROUND OF THE INVENTION

1. Field of the Invention

The present invention relates to a deoxyribonucleic acid (DNA) collection kit, particularly to a kit which comprises a foldable collection rod for collecting DNA and a collection bottle for placing the foldable collection rod to prolong the DNA preservation time. The present invention also relates to a method for collecting the DNA.

2. Description of the Prior Arts

The common method of DNA collection is to collect a tester's DNA with a collection rod, and then place the collection rod comprising collected DNA into an empty collection bottle or a collection bottle with a desiccant. Concentration of the collected DNA on the collection rod will be affected once the collection rod is collided with the collection bottle during transportation; besides, the accuracy of subsequent examination will be affected by DNA degradation caused by the desiccant.

Affinity between the collection rod and the collected DNA will affect DNA concentration when the collected DNA is dissolved in a subsequent solution. For example, if the affinity of the collected DNA adhered to the collection rod is stronger than to the solution, a large amount of the collected DNA will remain on the collection rod. For another example, material of the collection rod and specific functional group of the solution also affect degradation and quality of the collected DNA. As such, a technique that provides stable preservation of the collected DNA is needed.

### SUMMARY OF THE INVENTION

The conventional DNA preserving techniques have drawbacks of easily causing the DNA to degrade and affect the accuracy of the subsequent detection. To overcome the shortcomings, the present invention provides a DNA collection kit and a physical method for collecting a sample comprising DNA by using a foldable collection rod and a selective buffer for prolonging preservation time of the DNA, and increasing the accuracy of the subsequent examination.

The objective of the invention is to provide a DNA collection kit, comprising a foldable collection rod comprising a rod body comprising a front end, a back end, and a groove, wherein the groove is formed adjacent to the front end and surrounding an outer periphery of the rod body; a collection portion comprising an extension segment and a collection segment, wherein the extension segment is adjacent to the front end of the rod body and extends along an axis of the rod body, wherein the collection segment is located at an end of the extension segment that is distal from the rod body, wherein the collection segment is a soft cylinder formed with thermoplastic by a foaming process; a grip portion extending from the back end of the rod body, wherein the grip portion is a polygonal cylinder; a collection bottle comprising a bottle body which is a cylinder comprising an upward opening, and the bottle body comprising a receiving tube portion being tapered and connected to the opening, wherein the receiving tube portion has a receiving space which is wider at an end proximal to the opening and narrower at an opposite end for receiving the collection portion; a cap which is connected to the bottle body and corresponds to the opening of the bottle body, and a buckled portion formed around an inner periphery of the cap and correspondingly closing the opening of the bottle body; and a buffer comprising ethylenediamine tetraacetic acid (EDTA) at a concentration from 20 mM to 60 mM filled within the receiving tube portion of the bottle body.

Preferably, the grip portion is a hexahedron.

Preferably, the rod body is a cylindrical rod.

Preferably, the thermoplastic of the collection segment of the collection portion is selected from the group consisting nylon, polystyrene (PS), polypropylene (PP), polyurethane (PU), polystyrene (PS), polyesters and combinations thereof.

More preferably, the collection segment is a foamed nylon swab.

Preferably, the shape of the collection segment is a cylinder, a cone, a water drop, or a ball shape.

Preferably, the groove is a trapezoid groove surrounding the outer periphery of the rod body, and a side of the groove distal from the front end is a vertical plane.

Preferably, the bottle body has a flange around an outer periphery of the bottle body and near the opening, and the buckled portion of the cap has a ring formed around an inner edge of the cap, wherein the inner diameter of the ring is smaller than an outer diameter of the flange, and the ring of the cap correspondingly buckles the flange.

Preferably, the cap has a sealing tube portion protruding from an inner side of the cap and corresponding to the opening

Preferably, the cap is integrally connected to the flange of the bottle body with a connecting portion, which is a strip.

Preferably, the bottle body has a mark layer coated onto an exterior wall of the bottle body.

Preferably, the buffer further comprises a material selected from the group consisting of sodium chloride (NaCl), 2-amino-2-(hydroxymethyl)-1,3-propanediol hydrochloride (Tris-HCl), and sodium dodecyl sulfate (SDS).

More preferably, the buffer has a concentration of NaCl from 20 mM to 150 mM; the concentration of Tris-HCl from 10 mM to 60 mM; and the concentration of SDS from 0.1 w/v % to 10 w/v %.

In another aspect, the present invention also provides a method for using the above mentioned DNA collection kit, comprising the following steps: collecting DNA by the collection portion of the foldable collection rod of the DNA collection kit; breaking the foldable collection rod from the groove of the rod body to obtain a DNA-comprising collection portion; and putting the DNA-comprising collection portion into the collection bottle comprising the buffer to prolong the preservation time of the DNA, wherein the buffer comprises ethylenediamine tetraacetic acid (EDTA) at a concentration from 20 mM to 60 mM.

Preferably, the collection segment of the collection portion of the foldable collection rod is formed with thermoplastic by a foaming process, wherein the thermoplastic is selected from the group consisting of nylon, polystyrene (PS), polypropylene (PP), polyurethane (PU), polystyrene (PS), polyesters and combinations thereof. Preferably, the collection segment of the collection portion of the foldable collection rod is in shape of a cylinder, a cone, a water drop, or a ball.

Preferably, the buffer is further selected from the group consisting of sodium chloride (NaCl), 2-amino-2-(hydroxymethyl)-1,3-propanediol hydrochloride (Tris-HCl), and sodium dodecyl sulfate.

More preferably, a concentration of NaCl is from 20 mM to 150 mM; the concentration of Tris-HCl is from 10 mM to 60 mM; and the concentration of SDS is from 0.1 w/v % to 10 w/v %.

The advantages of the present invention are described as follows:

1. The rod body of the foldable collection rod in accordance with the present invention has a strengthened structure to enhance a hardness of the rod body, and the rod body comprises the grip portion at the back end for handling and avoiding slippage. The rod body is easy for applying force and hard to be deformed when rolling the rod body to collect the oral mucosa cell, and allows more oral mucosa cell to be adhered to the collection portion of the foldable collection rod and collects higher DNA concentration.

2. The foldable collection rod in accordance with the present invention comprises the groove for easily breaking the connection portion between the rod body and the collection portion. User can easily break the collection portion from the groove and detach the collection portion after collecting the oral mucosa cell. The user does not need to particularly apply force or use scissors to separate the rod body and the collection portion.

3. The collection segment of the collection portion of the rod body in accordance with the present invention is a foamed cylinder swab that can adhere more mucosa cell to the collection portion and collect higher DNA concentration, and also increase the user's comfort.

4. The collection bottle in accordance with the present invention comprises a flat bottom so that the collection bottle can be placed stably. The bottle body is designed to be wider at the end proximal to the opening end and narrower at the opposite side that allows using less buffer to reach a higher liquid level in order to save the buffer.

5. A sound is generated when the cap is buckled to the flange of the bottle body, for ensuring that the cap is buckled to the opening steadily and seamlessly, and to increase the effect of seal and preservation.

6. The method of collection DNA uses specific concentration of EDTA to avoid DNA degradation. The collection segment of the collection portion comprises a smooth surface that is densely multiporous, not only increasing the collection surface area of the collection segment of the collection portion to collect more subjects' mucosa cell, but also reducing subjects' discomfort. Beside, the ingredient of the buffer can reduce the adhesive of the sample to the collection segment of the collection portion in order to increase the DNA to be dissolved in the buffer, and increase the DNA concentration obtained in subsequent processes.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a DNA collection kit comprising a foldable collection rod, a collection bottle and a buffer.

FIG. 2 is a perspective view of the foldable collection rod in FIG. 1.

FIG. 3 is a partially-enlarged side view of the foldable collection rod in FIG. 1.

FIG. 4A is a perspective view of the collection bottle in FIG. 1, shown in an open status.

FIG. 4B is a perspective view of the collection bottle, shown in a closed status.

FIG. 5 is a cross-sectional view of the collection bottle.

FIG. 6 illustrates the use of the foldable collection rod of the DNA collection kit.

FIGS. 7A and 7B show operational views of the foldable collection rod when using the DNA collection kit.

FIG. 8 illustrates the use of the collection portion of the foldable collection rod put into the collection bottle.

FIG. 9 illustrates three steps for using DNA collection kit.

FIG. 10 illustrates a result of DNA agarose gel electrophoresis, wherein the sample is collected from the subject by a nylon brush and a foamed nylon swab respectively, and sample 1 is placed at room temperature for 4 days, lanes 1, 2, 3 are nylon brush groups, lanes 4, 5, 6 are foamed nylon swab groups, and lanes 1, 4 are untreated groups; lanes 2, 5 use the conventional technique of desiccant to preserve the collection portion of the foldable collection rod; lanes 3, 6 use GST buffer to preserve the collection portion of the foldable collection rod, M is DNA marker, the arrow indicates the degradation of the DNA (also called smear), and the same meanings of line 1-6, M and arrow apply to the following FIG. 11 and FIG. 12.

FIG. 11 illustrates a result of DNA agarose gel electrophoresis, wherein the sample is collected from the subject by a nylon brush and a foamed nylon swab respectively, and the sample 2 is placed at low temperature for 4 days.

FIG. 12 illustrates a result of DNA agarose gel electrophoresis, wherein the sample is collected from the subject by a nylon brush and a foamed nylon swab respectively, and the sample 3 is placed at room temperature for 8 days.

### DETAILED DESCRIPTION OF THE PREFERRED EMBODIMENTS

### EXAMPLE 1

### The Composition of the DNA Collection Kit

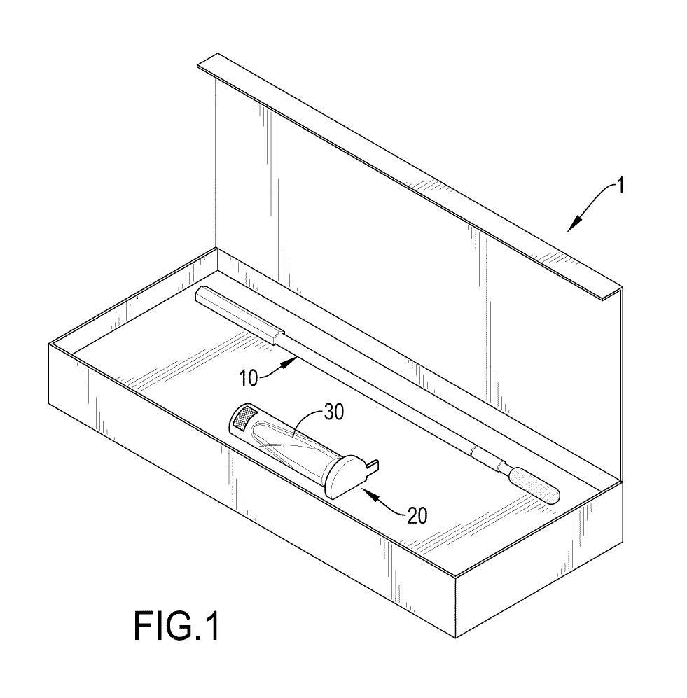

As shown in FIG. 1, the present invention provides a DNA collection kit 1 comprising a foldable collection rod 10, a collection bottle 20 and a buffer 30.

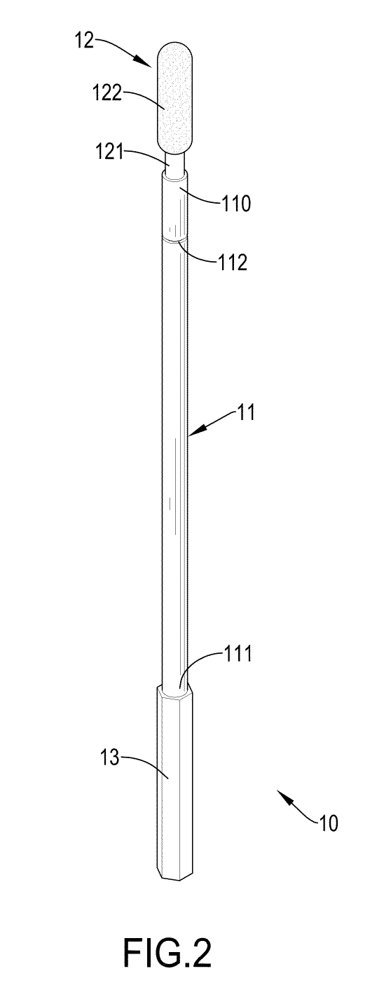

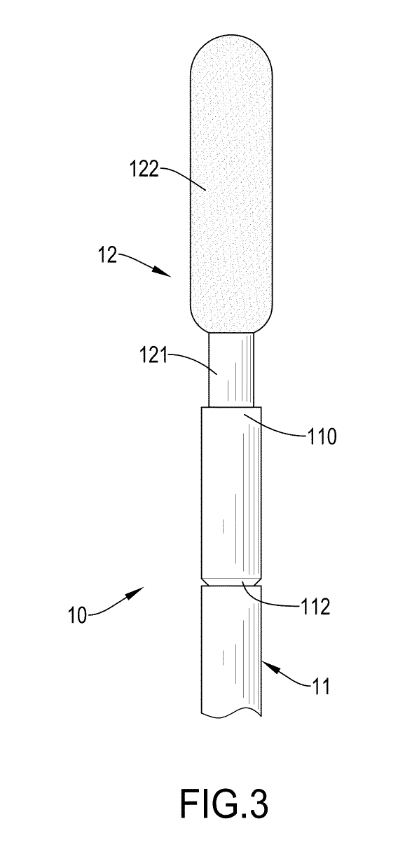

As shown in FIG. 2 and FIG. 3, the foldable collection rod 10 comprises a rod body 11, a collection portion 12 and a grip portion 13. The rod body 11 is a cylindrical rod comprising a front end 110 and a back end 111. A groove 112 is formed adjacent to the front end 110 and surrounds an outer periphery of the rod body 11. The groove 112 can be, but not limited to, a triangular groove, a rectangular groove, or a polygonal groove, and a side of the groove 112 distal from the front end 110 of the rod body 11 is a vertical plane. The rod body 11 with a high hardness is difficult to be bent, which helps the user force on the rod body 11 for increasing the amount of the collected oral mucosa cell.

The collection portion 12 comprises an extension segment 121 and a collection segment 122, wherein the extension segment 121 is adjacent to the front end 110 of the rod body 11 and extends along an axis of the rod body 11. The collection segment 122 is adjacent to an end of the extension segment 121 that is distal from the front end 110 of the rod body 11, wherein the collection segment 122 is formed with thermoplastic by a foaming process, wherein the thermoplastic was selected from the group consisting of nylon, polystyrene (PS), polypropylene (PP), polyurethane (PU), polystyrene (PS), polyesters and combinations thereof. In the instant example, the collection segment 122 is a foamed nylon swab formed as a cylinder in order to reduce the subject's discomfort and increase the amount of the collected DNA to achieve accurate test. The foamed nylon swab is purchased from Iron Will Biomedical Technology, Cat. No. 30221, wherein the length and the width of the collection portion 12 of the nylon swab are 22 millimeters (mm) and 7 mm, respectively, and the collection portion 12 is multiporous and formed with nylon by a foaming process. The foamed nylon swab is helpful for oral mucosa cell adhesion with higher DNA concentration, and increases comfort of the user.

The grip portion 13 extends from the back end 111 of the rod body 11, wherein the grip portion 13 is a polygonal cylinder, preferably a hexahedron. In order to handle the rod body 11 conveniently and avoid slippage when collecting the oral mucosa, the structure of the rod body 11 is strengthened, together with the rod body 11 formed as a cylinder and the grip portion 13 as a hexahedron.

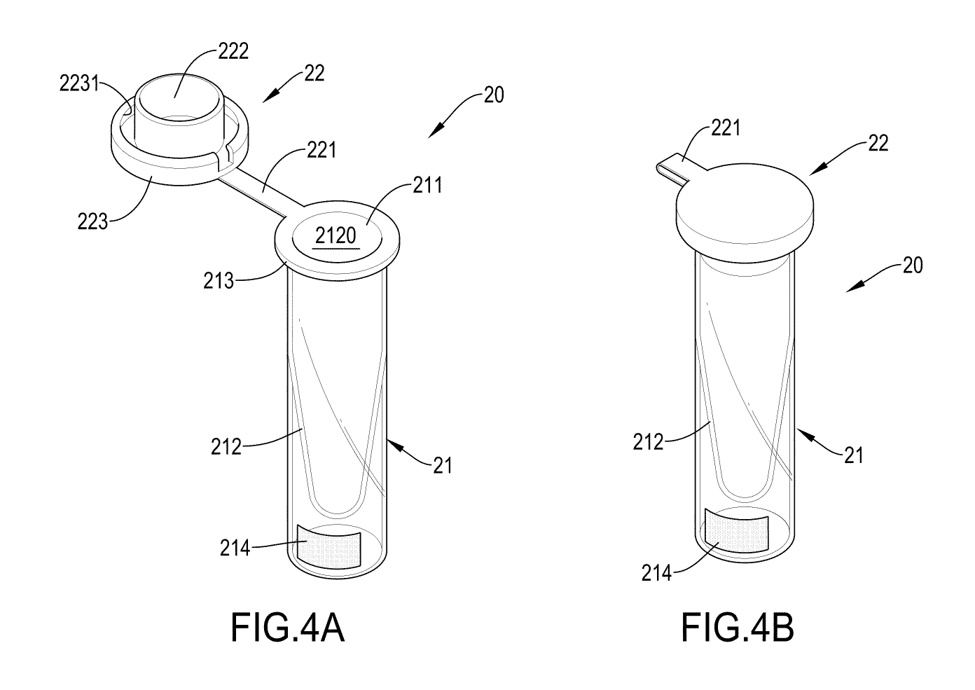

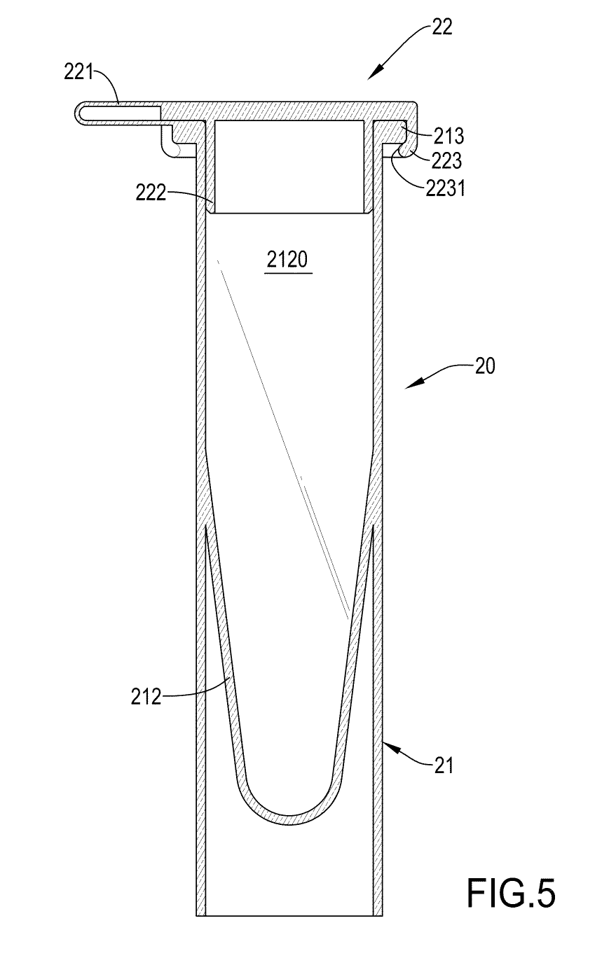

As shown in FIG. 4A, FIG. 4B and FIG. 5, the collection bottle 20 comprises a bottle body 21 and a cap 22. The bottle body 21 is a cylinder comprising an upward opening 211, and the bottle body 21 comprises a receiving tube portion 212 that is tapered and connected to the opening 211, wherein the receiving tube portion 212 comprises a receiving space 2120 which is wider at an end proximal to the opening and narrower at an opposite end for receiving the collection portion 12 of the foldable collection rod 10 and the buffer 30. The bottle body 21 has a flange 213 around an outer peripheral of the bottle body 21 and near the opening 211, and an exterior wall of the bottle body 21 is coated with a mark layer 214, preferably at the bottom of an exterior wall of the bottle body 21. A material of the mark layer 214 is same as the material of a signature portion of a credit card or an ordinary card on which writings by a water-based pen will not be dispersed, thereby eliminating the need for sticking any tag.

The cap 22 is connected to the flange 213 of the bottle body 21 with a connecting portion 221, which is a strip. The cap 22 can be correspondingly buckled to the opening 211 of the bottle body 21. The cap 22 comprises a sealing tube portion 222 which protrudes from an inner side of the cap 22 and the edge of the sealing tube portion 222 corresponds to the opening 211, wherein a buckled portion 223 is formed around an inner edge of the cap 22. The buckled portion 223 of the cap 22 comprises a ring 2231, wherein an inner diameter of the ring 2231 is smaller than an outer diameter of the flange 213.

When the cap 22 is closed on the bottle body 21, the sealing tube portion 222 is inserted to the opening 211 of the bottle body 21 in order to seal the receiving tube portion 212, and the buckled portion 223 is buckled with the flange 213 of the bottle body 21. When the ring 2231 is completely buckled to the flange 213 of the bottle body 21, the ring 2231 will be collided with the bottle body 21 and the flange 213, thereby producing a sound, as a notification that the cap 20 is buckled to the opening 211 of the bottle body 21 steadily and seamlessly.

As shown in FIG. 1, the buffer 30 comprises ethylenediamine tetraacetic acid (EDTA) at a concentration from 20 mM to 60 mM filled within the receiving tube portion 212 of the bottle body 21, wherein the buffer is further selected from the group consisting of sodium chloride (NaCl), 2-amino-2-(hydroxymethyl)-1,3-propanediol hydrochloride (Tris-HCl), and sodium dodecyl sulfate (SDS). The concentration of NaCl is from 20 mM to 150 mM, Tris-HCl from 10 mM to 60 mM, and SDS from 0.1 w/v % to 10 w/v %. In a preferable embodiment, the buffer 30 is GST buffer purchased from Geneaid™ company, comprising 25 mM ethylenediamine tetraacetic acid (EDTA), 100 mM sodium chloride (NaCl), 10 mM and pH 8.0 2-amino-2-(hydroxymethyl)-1,3-propanediol hydrochloride (Tris-HCl), and 0.5 w/v % sodium dodecyl sulfate (SDS) based on the total concentration of the buffer 30. In a preferable embodiment, the buffer 30 is guanidine isothiocyanate solution (GIT solution) comprising 4 M guanidine thiocyanate, 50 mM and pH 7.5 Tris-HCl, and 25 mM EDTA.

### EXAMPLE 2

### Steps of Using the DNA Collection Kit

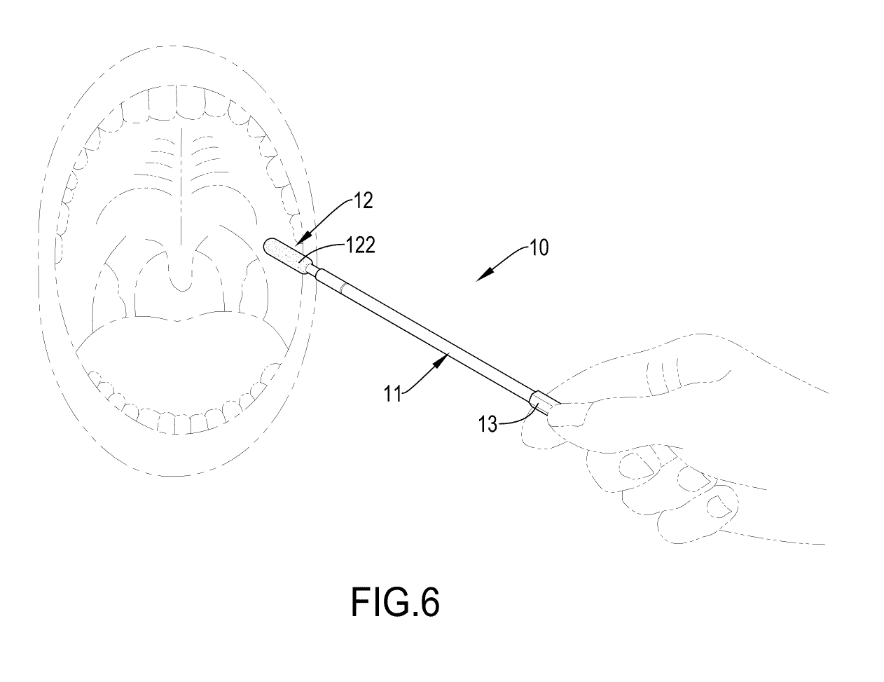

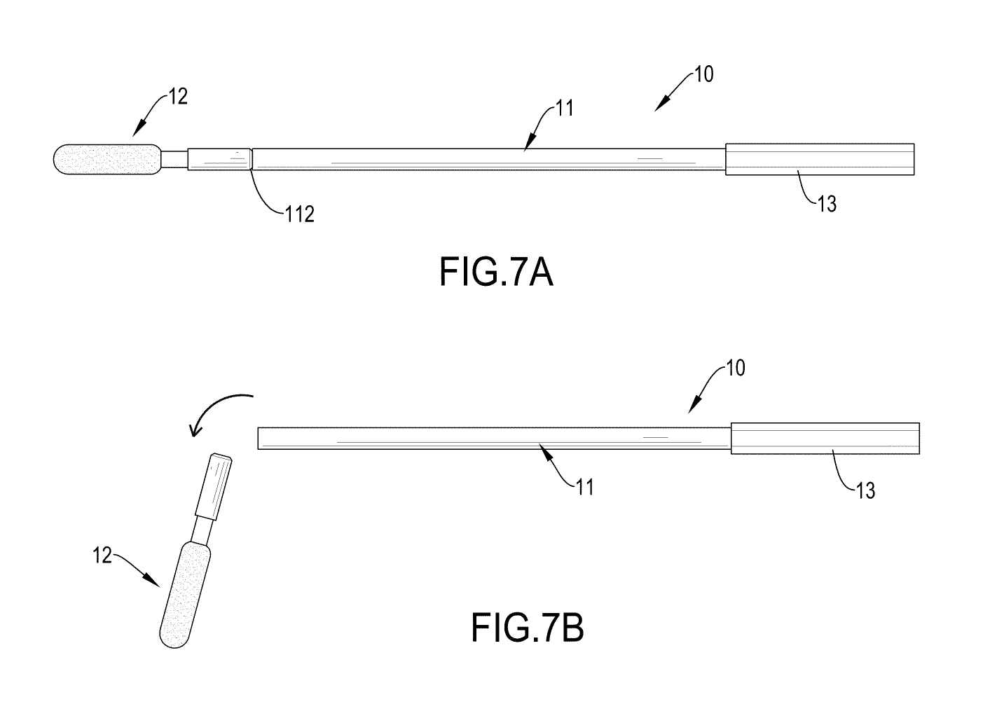

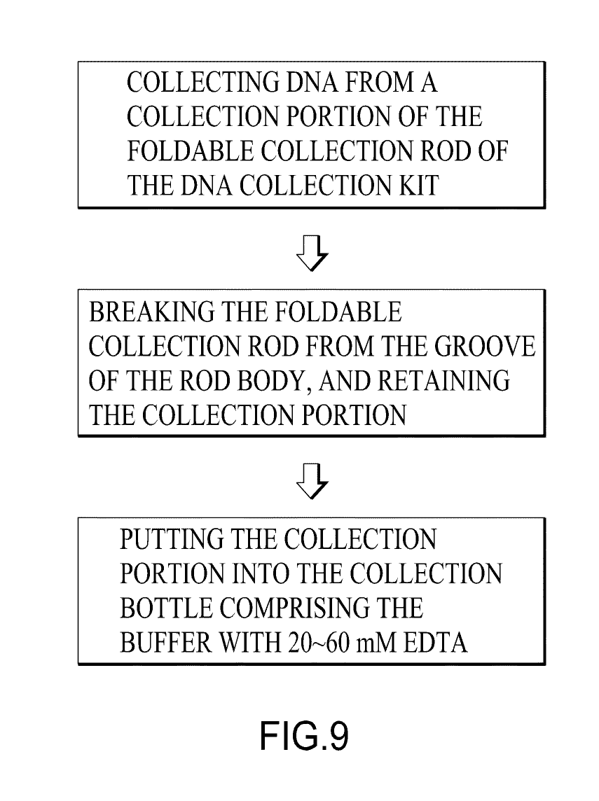

When the DNA collection kit of the present invention is in use, with reference to FIG. 6 and FIG. 9, an user can hold the grip portion 13 of the foldable collection rod 10 to collect oral mucosa cell from a subject by the collection segment 122 of the collection portion 12, for allowing the oral mucosa cell to be adhered to the collection segment 122 of the collection portion 12. With reference to, FIG. 7A, FIG. 7B and FIG. 9, after the oral mucosa cell is collected with the foldable collection rod 10, the collection portion 12 of the rod body 11 can be detached by breaking the rod body 11 from the groove 112. The oral mucosa cell of the subject is slightly scrapped for several times (preferable for 6 to 7 times) with the collection segment 122 of the collection portion 12 of the foldable collection rod 10.

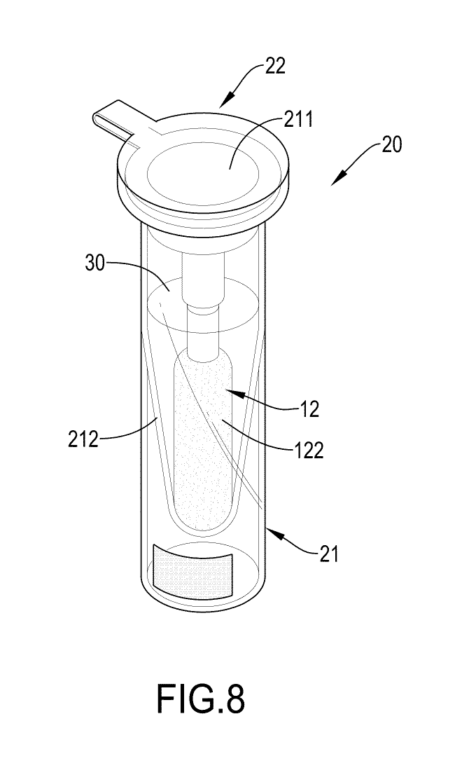

With reference to FIG. 8 and FIG. 9, the receiving tube portion 212 of the bottle body 21 can be filled with the buffer 30 and the collection portion 12, whereby the receiving tube portion 212 is wider at the end proximal to the opening 211 and narrower at the opposite end for using less buffer 30 to fully immerse the collection segment 122 of the collection portion 12 and saving the usage of the buffer 30. The bottle body 21 can be steadily put on a flat table by the flat bottom of the bottle body 21.

### EXAMPLE 3

### Effect of the Materials of the Collection Portion of the Collection Rod, the Kind of Buffer, Preservative Temperature and Preservative Time for DNA Concentration and Degradation

The oral mucosa cell was collected with the collection segment 122 of the collection portion 12 of the foldable collection rod 10, then the rod body 11 was broken from the groove 112 of the rod body 11, and the collection portion 12 comprising the subject's DNA was put into the empty collection bottle 20, the collection bottle 20 comprising a desiccant (purchased from ISOHELIX, LTD), or the collection bottle 20 with the buffer 30, for 2 days, 4,days, and 8 days at low temperature (4° C.) and room temperature (25° C.), and then the DNA was extracted by the following steps:

(a) the collection segment 122 of the collection portion 12 comprising the subject's DNA was put into a microcentrifuge tube containing 500 μL GST buffer and 20 μL protease K and heated at 60° C. for 10 minutes (mins), then the collection portion 12 was removed and discarded, and 500 μL GST buffer was added into the collecting bottle 20 and shaken for 10 minutes under 60° C. to destroy cell membranes and nuclei;

(b) 500 μL absolute ethanol was added and shaken evenly to form an extracting solution, and the extracting solution was transferred to a GD column (purchased from Geneaid™), and then centrifuged at 14,000×g to 16,000×g for 1 minute (min);

(c) 100 μL pre-heated eluted buffer or Tris-EDTA (TE) buffer was added and centrifuging at 14,000×g to 16,000×g for 30 seconds to obtain the extracted DNA; and

(d) concentration of the extracted DNA was analyzed by staining with ethidium bromide (EtBr) and purity of the extracted DNA was analyzed based on a ratio of DNA absorbance at 260 nm to 280 nm (OD₂₆₀/OD₂₈₀).

A method for analyzing concentration of the extracted DNA by staining with the EtBr comprised the following steps:

(a) a DNA marker was run with a known concentration and the extracted DNA with an unknown concentration by agarose gel electrophoresis followed by staining with the EtBr, and photographing in an ultraviolet (UV) light box; and

(b) concentration, purity and degree of degradation of the extracted DNA were calculated by comparing intensity of the DNA marker with the extracted DNA. The purity of the extracted DNA was determined by a value of the absorbance ratio of 260 nm to 280 nm. A value of OD₂₆₀/OD₂₈₀ of extracted DNA equal to 1.8 indicated high purity of DNA, whereas a value of extracted DNA less than 1.8 indicated low purity of DNA. The degree of DNA degradation was determined by a ratio of DNA pixels of a completed DNA (a major band of DNA) to DNA pixels of a degraded DNA (a smeared DNA), wherein the pixels of the completed DNA and the pixels of the degraded DNA were calculated by an Image J software. Higher value of the ratio indicated more complete DNA was preserved.

TABLE 1
Concentration and purity of the extracted DNA at different
temperatures (4° C., 25° C.) for different days
(2 days, 4 days, and 8 days).

| | | RT (25° C.) | LT (4° C.) | RT (25° C.) |
|---|---|---|---|---|
| | | 2 days | 4 days | 8 days |
| | | sample 1 | sample 2 | sample 3 |
| | | conc./purity | conc./purity | conc./purity |
| Nylon | untreated³ | 145/2.09 | 40/2.15 | 25/2.01 |
| brush¹ | desiccant | 51/2.04 | 75/2.01 | 135/2.15 |
| | GST buffer | 31/1.97 | 21/2.10 | 60/2.11 |
| foamed | untreated³ | 172/2.09 | 217/2.13 | 135/2.15 |
| nylon | desiccant | 181/2.08 | 81/2.07 | 274/2.14 |
| swab² | GST buffer | 184/2.05 | 144/2.11 | 391/2.07 |

conc.: concentration;
RT: room temperature;
LT: low temperature

1. The collection segment of the collection portion comprised multiple bristles which were arranged at equidistance and perpendicular to an axis of the foldable collection rod to allow a surface of the collection segment to be irregular. The nylon brush was purchased from Iron Will Biomedical Technology, Cat. No. 30021, wherein the length and the width of the collection segment of the collection portion of the nylon brush are 20 mm and 7 mm, respectively, and the collection segment of the collection portion comprises multiple bristles arranged equidistant from each other and perpendicular to the foldable collection rod.

2. The collection segment 122 of the collection portion 12 was multiporous, formed with a nylon treated by foaming process, which allowed a surface of the collection segment 122 to be smooth and in shape of a cylinder or cone.

3. “Untreated” indicated that the collection segment 122 of the collection portion 12 comprising the oral mucosa cell was not treated with the desiccant or GST buffer, and was directly placed at different temperatures for different days as shown in Table 1.

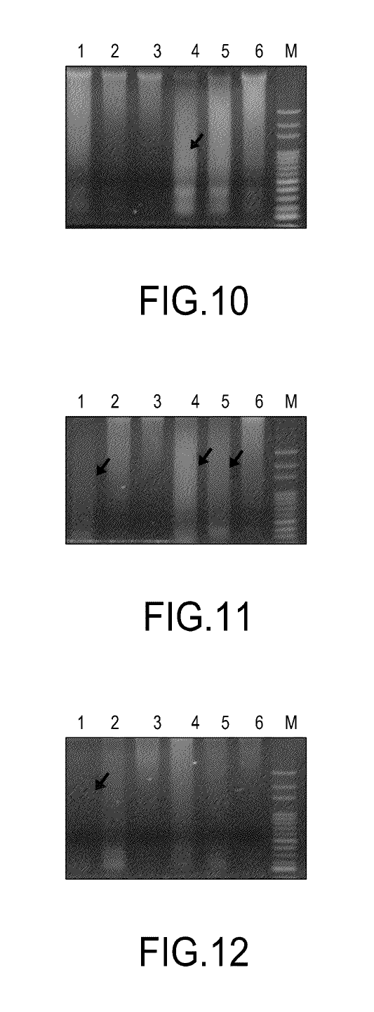

As shown in FIG. 10 and Table 1, the extracted DNA obtained from the foamed nylon swabs and from the nylon brushes were respectively put into the empty collection bottle (untreated group), the collection bottle with a desiccant, and the collection bottle 20 with the buffer 30 (GST buffer group), and then they were all placed at room temperature (25° C.) for 2 days. The result shows that higher concentration of the extracted DNA could be obtained from the untreated, the desiccant and the GST buffer group of the foamed nylon swab.

TABLE 2
The degradation of the extracted DNA at different temperatures (4° C.,
and 25° C.) for different days (2 days, 4 days, and 8 days).

| | | RT (25° C.) | LT (4° C.) | RT (25° C.) |
|---|---|---|---|---|
| | | 2 days | 4 days | 8 days |
| | | sample 1 | sample 2 | sample 3 |
| | | degradation | degradation | degradation |
| nylon brush | untreated | 1.5568 | 0.4733 | 3.2779 |
| | desiccant | 3.1411 | 1.8739 | 1.4268 |
| | GST buffer | 3.0440 | 4.0968 | 3.5681 |
| foamed nylon | untreated | 0.3163 | 0.5612 | 2.0581 |
| swab | desiccant | 0.3625 | 0.5574 | 2.0660 |
| | GST buffer | 1.6227 | 3.4140 | 6.4024 |

conc.: concentration,
RT: room temperature,
LT: low temperature

As shown in FIG. 11, the extracted DNA obtained from the foamed nylon swabs and from the nylon brushes were respectively put into the empty collection bottle (untreated group), the collection bottle with a desiccant, and the collection bottle 20 with the buffer 30 (GST buffer group), and then they were all placed at low temperature (4° C.) for 4 days, the extracted DNA was degraded in the untreated group and the desiccant group of both of the nylon brush and the foamed nylon swab.

As shown in FIG. 12, the extracted DNA obtained from the foamed nylon swabs and from the nylon brushes were respectively put into the empty collection bottle (untreated group), the collection bottle with a desiccant, and the collection bottle 20 with the buffer 30 (GST buffer group), and then they were all placed at room temperature (25° C.) for 8 days, the extracted DNA was degraded in the untreated group of both of the nylon brush and the foamed nylon swab.

From the above, preserving the collection portion 12 at low temperature (4° C.) avoided DNA degradation and prolonged the DNA preservation time. The foamed nylon swab collected much higher DNA concentration than the nylon brush. As shown in table 2, the foamed nylon swab with the GST buffer group treatment could collect more completed DNA than the nylon brush with the desiccant or the untreated group treatment.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A deoxyribonucleic acid (DNA) collection kit, comprising:
a foldable collection rod comprising
a rod body comprising a front end, a back end, and a groove, wherein the groove is formed adjacent to the front end and surrounding an outer periphery of the rod body;
a collection portion comprising an extension segment and a collection segment, wherein the extension segment is adjacent to the front end of the rod body, and extends along an axis of the rod body, wherein the collection segment is located at an end of the extension segment that is distal from the rod body, wherein the collection segment is a soft cylinder formed with thermoplastic by a foaming process;
a grip portion extending from the back end of the rod body, wherein the grip portion is a polygonal cylinder;
a collection bottle comprising
a bottle body being a cylinder comprising an upward opening, the bottle body comprising a receiving tube portion being tapered and connected to the opening, wherein the receiving tube portion has a receiving space which is wider at an end proximal to the opening and narrower at an opposite end for receiving the collection portion;
a cap connected to the bottle body and corresponds to the opening of the bottle body, and a buckled portion formed around an inner periphery of the cap and correspondingly closing the opening of the bottle body; and
a buffer comprising ethylenediamine tetraacetic acid (EDTA) at a concentration from 20 mM to 60 mM filled within the receiving tube portion of the bottle body.

2. The DNA collection kit according to claim 1, wherein the grip portion is a hexahedron.

3. The DNA collection kit according to claim 1, wherein the rod body is a cylindrical rod.

4. The DNA collection kit according to claim 1, wherein the thermoplastic of the collection segment is selected from the group consisting of nylon, polystyrene (PS), polypropylene (PP), polyurethane (PU), polystyrene (PS), polyesters and combinations thereof.

5. The DNA collection kit according to claim 4, wherein the collection segment is a foamed nylon swab.

6. The DNA collection kit according to claim 1, wherein the shape of the collection segment is a cylinder, a cone, a water drop, or a ball shape.

7. The DNA collection kit according to claim 1, wherein the groove is a trapezoid groove surrounding the outer periphery of the rod body, and a side of the groove distal from the front end is a vertical plane.

8. The DNA collection kit according to claim 1, wherein the bottle body has a flange around an outer periphery of the bottle body and near the opening, and the buckled portion of the cap has a ring formed around an inner edge of the cap, wherein an inner diameter of the ring is smaller than an outer diameter of the flange, and the ring of the cap correspondingly buckles the flange.

9. The DNA collection kit according to claim 1, wherein the cap has a sealing tube portion protruding from an inner side of the cap and corresponding to the opening.

10. The DNA collection kit according to claim 1, wherein the cap is integrally connected to the flange of the bottle body with a connecting portion, which is a strip.

11. The DNA collection kit according to claim 1, wherein the bottle body has a mark layer coated onto an exterior wall of the bottle body.

12. The DNA collection kit according to claim 1, wherein the buffer further comprises a material selected from the group consisting of sodium chloride (NaCl), 2-amino-2-(hydroxymethyl)-1,3-propanediol hydrochloride (Tris-HCl), and sodium dodecyl sulfate (SDS).

13. The DNA collection kit according to claim 12, wherein the NaCl has a concentration from 20 mM to 150 mM; the Tris-HCl has a concentration from 10 mM to 60 mM; and the SDS has a concentration from 0.1 w/v % to 10 w/v %.

14. A method for using a DNA collection kit as claimed in claim 1, comprising following steps:
collecting DNA by the collection portion of the foldable collection rod of the DNA collection kit;
breaking the foldable collection rod from the groove of the rod body to obtain a DNA-comprising collection portion; and
putting the DNA-comprising collection portion into the collection bottle comprising the buffer to prolong the preservation time of the DNA, wherein the buffer comprises ethylenediamine tetraacetic acid (EDTA) at a concentration from 20 mM to 60 mM.
